(19) United States
(12) Patent Application Publication
(10) Pub. No.: US 2008/0097350 A1
(43) Pub. Date: Apr. 24, 2008

(54) MEDICAL DEVICES

(76) Inventors: Barbara A. Bell, Sudbury, MA (US); Christopher J. Elliott, Hopkinton, MA (US); Paul D. Dicarlo, Middleboro, MA (US); Raymond J. Lareau, Westford, MA (US); Victor Efimovich Minaker, Moscow (RU); Vasiliy Gennadievich Abashkin, Saint-Petersburg (RU); Valery Valentinovich Pavlov, Saint-Petersburg (RU); Valentin Vassilievich Titov, Moscow (RU); Pavel Mikhailovich Karavaev, Saint-Petersburg (RU); Leonid Malinin, Newton, MA (US); Andrey Viacheslavovich Efimov, Saint-Petersburg (RU); Alexander Vladimirovich Kudryavtsev, Moscow (RU)

Correspondence Address: FISH & RICHARDSON PC, P.O. BOX 1022, MINNEAPOLIS, MN, 55440-1022, US

(21) Appl. No.: 11/428,979

(22) Filed: Jul. 6, 2006

(51) Int. Cl. A61M 25/14

(52) U.S. Cl. 604/266; 604/264

(57) ABSTRACT

Medical devices, as well as related systems and methods, are disclosed.

### TECHNICAL FIELD

This disclosure generally relates to medical devices, as well as related systems and methods.

### BACKGROUND

Medical devices with multiple internal lumens can be placed within veins, arteries, and other body lumens. Examples of medical devices with multiple lumens include catheters and stents.

### SUMMARY

In one aspect, the invention generally relates to a catheter that includes an elongated tubular element having a wall, and a septum attached to the inner surface of the wall in at least two places so that the septum and wall form at least two lumens in the catheter. A length of the septum is greater than or equal to a maximum distance between two points on the inner surface of the wall.

In another aspect, the invention generally relates to a catheter that includes an elongated tubular element having a wall, and a septum attached to the inner surface of the wall in at least two places so that the septum and wall form at least two lumens in the catheter. The catheter also includes an anti-adhesive material supported by the inner surface of the wall.

In a further aspect, the invention generally relates to a catheter that includes an elongated tubular element having a wall, and a septum attached to the inner surface of the wall in at least two places so that the wall and the septum form at least two lumens. The catheter also includes a material supported by the outer surface of the wall.

In an addition aspect, the invention generally relates to a catheter that includes an elongated tubular element having a wall, and a septum attached to the inner surface of the wall in at least two places so that the wall and the septum form at least two lumens. The wall includes a protrusion that extends radially inward.

In a further aspect, the invention generally relates to a catheter that includes an elongated tubular element having a wall, and a septum attached to the inner surface of the wall in at least two places, where the article is a catheter. The wall has a recess that extends radially outward.

Embodiments can include one or more of the following features.

The septum can be attached to the inner surface of the wall in two places.

In some embodiments, the ratio of the length of the septum to the maximum distance between two points on the inner surface of the wall can be at least 1:1. In certain embodiments, the ratio of the length of the septum to the maximum distance between two points on the inner surface of the wall can be at most 10:1.

The septum can include a polymer.

An anti-adhesive material can be supported by the inner surface of the wall.

A material can be supported by the outer surface of the wall.

The wall can include a protrusion that extends radially inward. In some embodiments, the length of the protrusion can be less than half a length of the septum. In certain embodiments, the length of the protrusion can be more than half a length of the septum

The wall can include a recess that extends radially outward. In some embodiments, the ratio of a depth of the recess to a maximum thickness of the wall can be at most 0.95:1. In certain embodiments, the ratio of a depth of the recess to a maximum thickness of the wall can be at least 0.05:1.

Embodiments of the invention can include one or more of the following advantages.

Medical devices with two or more internal lumens can be used to deliver and/or extract fluids from a body site. For example, the multiple lumens can be used to deliver nutrients, therapeutic agents such as pharmaceuticals, and cleaning and/or irrigation fluids (e.g., water). One or more lumens can also be used for providing suction at the body site to withdraw material via the one or more lumens. Multiple lumen devices enable the flow of materials in opposite directions simultaneously within medical devices.

Multiple-lumen medical devices can be used during laparoscopic surgery to provide both suction and irrigation. Conventional medical devices provide a single lumen for applying suction and for transporting irrigation fluid. Only one of these functions can be performed at a particular time, and conventional medical devices typically alternate between applying suction and providing fluid. Conventional medical devices are flushed with cleaning solution when switching between suction and irrigation functions. Multiple-lumen medical devices can be used to provide suction and irrigation simultaneously via one or more internal lumens dedicated to each function, thereby ensuring freedom from contamination, less use of cleaning solution, and more efficient operation during surgical procedures.

Medical devices with two or more internal lumens can be directed with guidewires to specific target sites within the body. One of the internal lumens can be dedicated to providing a passageway for guidewire insertion, while the other internal lumens can be used to provide other functions (e.g., transport of materials to/from body sites).

Multiple-lumen medical devices can include a relatively inelastic outer coating that substantially reduces radial expansion of the devices when pressurized fluid is directed to flow therein. Restriction of radial expansion can be used to prevent trauma to a body lumen that might otherwise result from device expansion. Medical devices with inelastic coatings are particularly well suited for deployment within body lumens having a relatively small cross-sectional area, as these body lumens can sometimes be more delicate and less tolerant to applied radial forces than larger body lumens.

Fluid pressure and/or flow rate can be used in multiple-lumen medical devices to control activation of one or more internal septa—that is, to control a direction and magnitude of force applied to one or more internal septa to cause displacement of the septa, thereby changing the cross-sectional area of one or more internal lumens. The flow rate of a fluid within a lumen is dependent on the lumen's effective cross-sectional area. By changing fluid pressure in one or more lumens to cause displacement of one or more septa within the device, fluid flow rates in other lumens can be changed in a controlled manner. Therefore, for example, the rate at which materials such as water, therapeutic agents, nutrients, and other materials can be delivered to a body site can be controlled. Alternatively, or in addition, suction pressure at a body site can be controlled in the same fashion.

Occlusions that form in one or more lumens of a medical device can be reduced in size or removed by manipulating device lumens. For example, during use, lumens in medical devices can become occluded with debris and deposits carried by fluids flowing therein. Displaceable septa in multi-lumen medical devices allow at least partial unclogging of these lumens. During use, a fluid can be flowed at high pressure in an unclogged lumen to displace a septum within a medical device. The displaced septum exerts a force on the occlusion, compressing the occluding material against a wall of the occluded lumen. A fluid can then be flowed through the previously occluded lumen to re-open a passageway therein.

Other features and advantages of the invention will be apparent from the description, drawings, and claims.

### DESCRIPTION OF DRAWINGS

FIG. 1A is a plan view of an embodiment of a catheter with multiple lumens.

FIG. 1B is a side view of the catheter of FIG. 1A.

FIG. 2A is a side view of an embodiment of a catheter with a relatively inelastic septum.

FIG. 2B is a side view of the catheter of FIG. 2A with fluid flowing in one lumen.

FIG. 3A is a side view of an embodiment of a catheter with a relatively elastic septum.

FIG. 3B is a side view of the catheter of FIG. 3A with fluid flowing in one lumen.

FIG. 4A is a side view of an embodiment of a catheter with a septum attached at two non-opposed positions.

FIG. 4B is a side view of the catheter of FIG. 4A with fluid flowing in one lumen.

FIG. 5 is a side view of an embodiment of a catheter with an anti-adhesion coating.

FIG. 6A is a side view of an embodiment of a catheter with a plurality of protrusions extending radially inward.

FIG. 6B is a side view of the catheter of FIG. 6A with fluid flowing in one lumen.

FIG. 7 is a side view of an embodiment of a catheter with a plurality of protrusions having cross-sectional shapes that feature undercut regions.

FIG. 8A is a side view of an embodiment of a catheter with a single protrusion.

FIG. 8B is a side view of the catheter of FIG. 8A with fluid flowing in one lumen.

FIG. 9A is a side view of an embodiment of a catheter with two protrusions.

FIG. 9B is a side view of the catheter of FIG. 9A with fluid flowing in one lumen.

FIG. 10A is a side view of an embodiment of a catheter with a protrusion attached to a septum.

FIG. 10B is a side view of the catheter of FIG. 10A with fluid flowing in a first lumen.

FIG. 10C is a side view of the catheter of FIG. 10A with fluid flowing in second and third lumens.

FIG. 11A is a side view of an embodiment of a catheter with a plurality of recesses extending radially outward.

FIG. 11B is a side view of the catheter of FIG. 11A with fluid flowing in one lumen.

FIG. 12 is a side view of an embodiment of a catheter with a coating disposed on an outer surface of the catheter.

FIGS. 13A-D show side views of an embodiment of a catheter with an occlusion.

FIG. 14 is a schematic diagram of a hub that connects a multiple-lumen catheter to separate outlet tubes.

Like reference symbols in the various drawings indicate like elements.

### DETAILED DESCRIPTION

This disclosure relates to medical devices such as catheters that have multiple internal lumens. The cross-sectional areas of the multiple internal lumens can be changed, for example, by flowing fluids through one or more of the lumens.

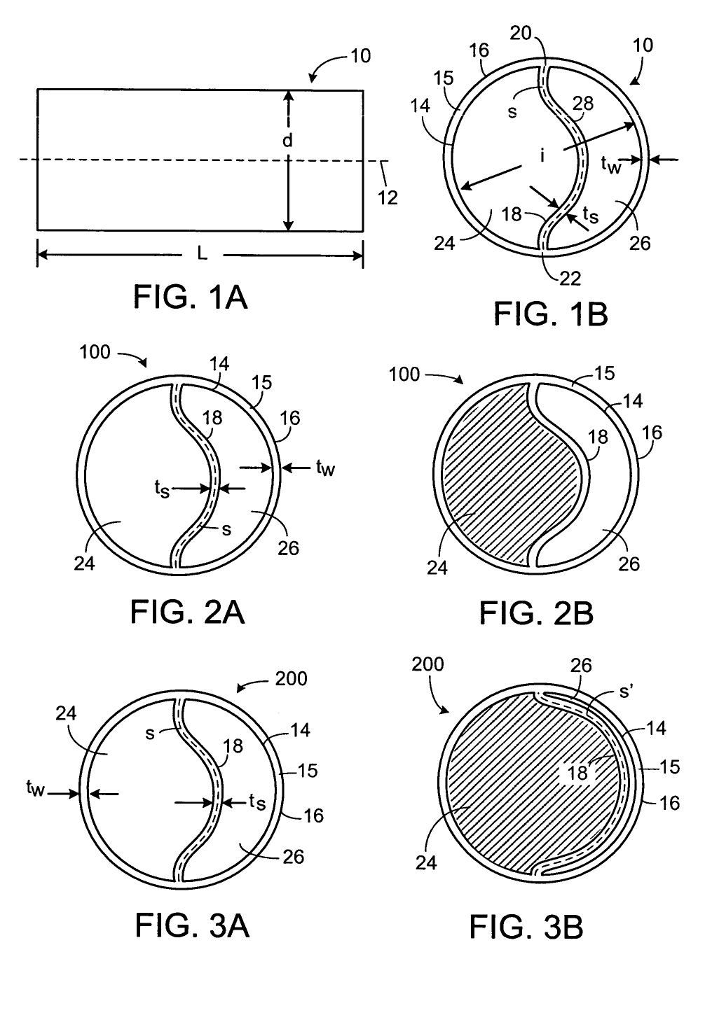

FIGS. 1A and 1B show plan and side views, respectively, of a catheter 10 that has multiple internal lumens. Catheter 10 has a tubular body of length L measured in a direction parallel to longitudinal axis 12, and an outer diameter d measured in a radial direction perpendicular to axis 12. Catheter 10 has a wall 15 with an inner surface 14 and an outer surface 16, and a thickness t_{w} of catheter material forming wall 15. A septum 18 is attached to inner surface 14 at positions 20 and 22. Septum 18 is formed from a thickness tₛ of septum material. A length of septum 18 is denoted by s, the length of dotted line 28 in FIG. 1B (corresponding to the length of septum 18 when septum 18 is not bent or stretched). Septum 18 and wall 15 form two lumens 24 and 26 in catheter 10. The cross-sectional area of lumens 24 and 26 depends on the position of septum 18.

In general, the length L of catheter 10 can be selected as desired. In some embodiments, L can be chosen according to a particular intended use for catheter 10, e.g., according to one or more physiological properties of a body lumen where catheter 10 will be inserted. In certain embodiments, L can be 1 mm or larger (e.g., 5 mm or larger, 10 mm or larger, 20 mm or larger, 30 mm or larger, 40 mm or larger). In some embodiments, L can be 300 cm or smaller (e.g., 20 cm or smaller, 100 cm or smaller, 50 cm or smaller, 1 cm or smaller). As an example, in some embodiments in which catheter 10 is an ocular drainage shunt, L can be 1 mm. As another example, in certain embodiments in which catheter 10 is employed in endoscopic use, L can be 300 cm.

In general, the outer diameter d of catheter 10 can be selected as desired. For example, the outer diameter d can be chosen so that the cross-sectional areas of each of the lumens in catheter 10 are sufficient to provide adequate fluid flow to or from a body site. In some embodiments, d can be 0.03 inch or larger (e.g., 0.05 inch or larger, 0.06 inch or larger, 0.07 inch or larger, 0.08 inch or larger). In certain embodiments, d can be 0.5 inch or smaller (e.g., 0.3 inch or smaller, 0.2 inch or smaller, 0.1 inch or smaller). As an example, in some embodiments in which catheter 10 is an ocular shunt, d can be 0.007 inch.

In general, the inner diameter i of catheter 10 can be selected as desired. For example, in some embodiments, i can be 0.03 inch or larger (e.g., 0.05 inch or larger, 0.06 inch or larger, 0.07 inch or larger, 0.08 inch or larger). In certain embodiments, i can be 0.5 inch or smaller (e.g., 0.3 inch or smaller, 0.2 inch or smaller, 0.1 inch or smaller). As an example, in some embodiments in which catheter 10 is an ocular shunt, i can be 0.005 inch.

The thickness t_{w} of catheter material forming wall 15 can generally be selected as desired to impart particular flexibility and elasticity to lumen walls. In some embodiments, for example, t_{w} can be 0.001 inch or larger (e.g., 0.002 inch or larger, 0.003 inch or larger, 0.005 inch or larger, 0.007 inch or larger, 0.01 inch or larger). In certain embodiments, t_{w} can be 0.05 inch or smaller (e.g., 0.02 inch or smaller, 0.01 inch or smaller, 0.005 inch or smaller, 0.001 inch or smaller).

The thickness tₛ of material that forms septum 18 can generally be the same as t_{w} or different from t_{w}. For example, in some embodiments, tₛ can be less than t_{w} so that, depending upon the catheter and septum materials, septum 18 can be more elastic than wall 15 of catheter 10 (e.g., elastic deformation of septum 18 under an applied force is larger than elastic deformation of wall 15). In some embodiments, tₛ can be 0.001 inch or larger (e.g., 0.002 inch or larger, 0.003 inch or larger, 0.005 inch or larger, 0.007 inch or larger, 0.01 inch or larger). In certain embodiments, tₛ can be 0.05 inch or smaller (e.g., 0.02 inch or smaller, 0.01 inch or smaller, 0.005 inch or smaller, 0.001 inch or smaller).

In general, the length s of septum 18 is greater than or equal to a maximum distance between two points on inner surface 14 of wall 15. In FIGS. 1A and 1B, catheter 10 has a circular cross-section, and the maximum distance between two points on inner surface 14 of wall 15 corresponds to the inner diameter i of catheter 10. For such a catheter, the length s of septum 18 is generally greater than or equal to the inner diameter i of catheter 10. In some embodiments, however, one or more regions (e.g., the entire length) of the catheter may have a non-circular cross-section (e.g., square, triangular, trapezoidal, elliptical, semicircular, compound, rhomboid, semi-elliptical). In such embodiments, the length s of septum 18 is generally greater than or equal to the maximum distance between two points on the inner surface of the catheter.

In some embodiments, a ratio of the length s of septum 18 to the maximum distance between two points on inner surface 14 of wall 15 is at least 1:1 (e.g., at least 2:1, at least 3:1, at least 4:1, at least 5:1). In certain embodiments, the ratio of the length s of septum 18 to the maximum distance between two points on inner surface 14 of wall 15 is at most 10:1 (e.g., at most 9:1, at most 8:1, at most 7:1, at most 6:1, at most 5:1).

Wall 15 of catheter 10 can generally be formed from any of a variety of materials. Often, wall 15 is formed of a polymer. Examples of polymers include thermoplastic polyurethanes (e.g., thermoplastic polyurethanes based on polyesters, polyethers, polycarbonates, and polysiloxanes such as Tecoflex®, Tecothane®, and Bionate®), polyamides (e.g., polyamide 12, polyamide 11, nylon, polyamide 6-12), polyether block amide elastomers (e.g., PEBAX®), and polyolefins (e.g., EVA, high density polyethylene, medium density polyethylene, low density polyethylene, SBS, and SIBS). Different catheter materials can be selected according to the intended use of catheter 10. For example, if catheter 10 is intended for use as a venous access device, wall 15 can be formed from materials such as polyurethanes and/or silicones. As another example, if catheter 10 is intended for use as a cannula, wall 15 can be formed from materials such as nylons and/or polyether block amides. Optionally, wall 15 can be formed from a mixture of materials (e.g., a mixture of two or more of the materials noted above.)

In some embodiments, catheter 10 can include various types of additives in the material that forms wall 15. For example, the material can include radiopaque materials such as bismuth-containing materials (e.g., bismuth trioxide, bismuth bicarbonate, bismuth oxychloride, and other bismuth-containing materials), metals (e.g., tungsten, platinum, silver, and other metals), alloys (e.g., tungsten-containing alloys, platinum-containing alloys, and other alloys), barium-containing materials (e.g., barium sulfate and other barium-containing materials), and other materials. Wall 15 can also include one or more materials to impart lubricity to catheter 10, such as various fluoropolymer oils, lubricating agents and/or other lubricating additives. Examples include perfluoroethylene, silicone oil, Teflon® flake, Teflon® powder and graphite.

In certain embodiments, the concentrations of additives can vary along the length of catheter 10 (e.g., additive concentrations can vary in a direction parallel to axis 12). For example, concentrations of radiopaque materials can vary to indicate specific locations along a catheter body. The marked locations can then be identified in x-ray images of the catheter as it is inserted or withdrawn from a body site. The markings can be used to ensure reproducible and accurate positioning of catheter 10 with respect to body sites.

Septum 18 can be formed from any of the materials discussed above in connection with wall 15, or from mixtures thereof. Additionally or alternatively, septum 18 can be formed of other materials, such as, for example, natural latex rubber and thermoplastic vulcinates (TPV). In certain embodiments, septum 18 can be formed from the same material used to form wall 15 (e.g., septum 18 can be integral with wall 15). In other embodiments, septum 18 can be formed from a material that is different from the material of wall 15. For example, in certain embodiments, septum 18 can be formed from a material that is more elastic than the material that forms wall 15.

In general, the shape of septum 18 can be selected as desired to provide a particular cross-sectional shape of lumens 24 and 26 within catheter 10. For example, in the embodiment shown in FIG. 1B, septum 18 has a wavy, undulating shape. In other embodiments, septum 18 can be arc-shaped, for example. In certain embodiments, septum 18 can include two linear portions joined at a cusp. In yet further embodiments, septum 18 can have other symmetric or asymmetric shapes.

During use, fluids can be directed to flow through lumens 24 and 26 of catheter 10. Fluids can include gases (e.g., nitrogen, oxygen, nitrous oxide, carbon dioxide, and other gases) and liquids (e.g., water). Fluids can also include solutions of one or more materials dissolved in one or more solvents (e.g., barium ions dissolved in water). The flow rate in a particular lumen varies according to the cross-sectional area of the lumen. Lumens with larger cross-sectional areas can support higher fluid flow rates than lumens with smaller cross-sectional areas.

The cross-sectional areas of lumens 24 and 26 can be changed by displacing septum 18 from its equilibrium position in FIG. 1B. Septum 18 can be displaced by changing the relative pressures of the fluids in lumens 24 and 26. For example, a fluid flowing at a high pressure in a lumen (e.g., lumen 24 or 26) exerts a force on septum 18 that tends to push septum 18 away from the flowing fluid. When septum 18 is positioned between two lumens (e.g., lumens 24 and 26), the pressures of fluids in these lumens can be controlled to adjust the displacement of septum 18. Thus, for example, the pressure of a fluid flowing in one lumen can be adjusted in order to change the flow rate of one or more fluids flowing in one or more other lumens.

The amount by which septum 18 is displaced by fluid pressure and the shape of the displaced septum depend in part upon the septum material. For example, in some embodiments, septum 18 can be formed from a flexible but relatively inelastic material. FIGS. 2A and 2B show side views of a catheter 100 without and with fluid flowing in lumen 24, respectively. A thickness tₛ of septum 18 is similar to a thickness t_{w} of wall 15 of catheter 100. Septum 18 is formed from a material that is sufficiently flexible so that, due to the pressure exerted by the fluid, septum 18 is displaced from its equilibrium position (shown in FIG. 2A) so that the cross-sectional area of lumen 24 is increased relative to FIG. 2A, and the cross-sectional area of lumen 26 is decreased relative to FIG. 2A. However, the material from which septum 18 is formed is generally relatively inelastic so that the length s of septum 18 does not change substantially under the influence of fluid pressure in lumen 24. In general, the thickness tₛ, length s, and elasticity of septum 18 are chosen such that septum 18 does not contact inner surface 14 of wall 15 when it is displaced in the direction of lumen 26. As a result, a different fluid can be directed to flow in lumen 26 even though the cross-sectional area of lumen 26 has been reduced.

In certain embodiments, septum 18 can be formed from a material that is both flexible and elastic, e.g., a material that deforms and stretches under the influence of applied fluid pressure. FIGS. 3A and 3B show side views of a catheter 200 without and with fluid flowing in lumen 24, respectively. In some embodiments, such as the embodiment shown in FIG. 3A, the thickness tₛ of septum 18 is similar to the thickness t_{w} of wall 15 of catheter 200. In other embodiments, tₛ can be less than t_{w} to provide a septum 18 that stretches more easily in response to an applied force. When a fluid at sufficiently high pressure is introduced into lumen 24, the fluid exerts a force on septum 18 that displaces septum 18 from its equilibrium position. As shown in FIG. 3B, the force applied to septum 18 can be sufficiently large that septum 18 contacts a portion of inner surface 14 of wall 15 in lumen 26. Septum 18 stretches in response to the applied fluid force on account of its elasticity. Thus, for example, a length s′ of septum 18 in FIG. 3B is larger than a length s of septum 18 in FIG. 3A.

When the fluid pressure in lumen 24 is reduced, elastic forces within septum 18 pull the septum material away from inner surface 14 in lumen 26, thereby preventing sticking of septum 18 to inner surface 14 and ensuring that blockage of lumen 26 due to septum adhesion does not occur.

The increase in cross-sectional area of lumen 24 in FIG. 3B is greater than the increase in cross-sectional area of lumen 24 in FIG. 2B, due to the larger displacement of septum 18 in FIG. 3B. Lumen 24 in FIG. 3B therefore supports a larger fluid flow rate than lumen 24 in FIG. 2B. In contrast, the cross-sectional area of lumen 26 in FIG. 3B is smaller than the cross-sectional area of lumen 26 in FIG. 2B. As shown in FIG. 3B, in certain embodiments, lumen 26 can be nearly sealed by septum 18. As a result, the fluid flow capacity of lumen 26 in FIG. 3B is less than the fluid flow capacity of lumen 26 in FIG. 2B.

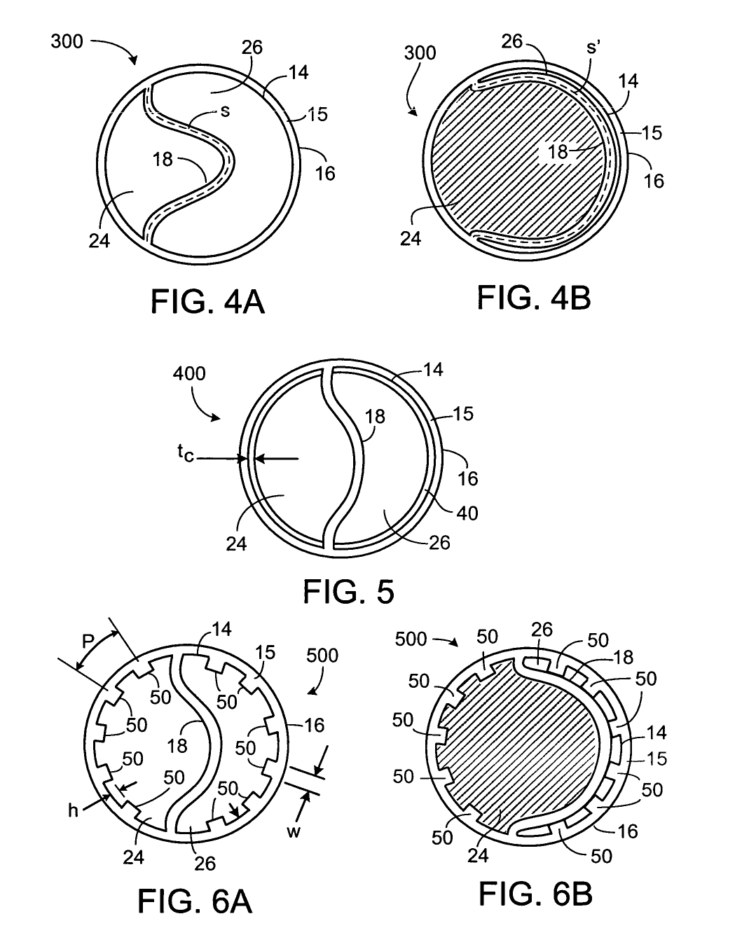

In certain embodiments, septum 18 can be attached to inner wall 14 at points that are not symmetrically opposed. For example, FIGS. 4A and 4B show side views of a catheter 300 without and with fluid flowing in lumen 24, respectively. The thicknesses t_{w} and tₛ of wall 15 and septum 18 can be the same or different, and can have the values discussed previously. Wall 15 and septum 18 can be formed from the materials discussed in connection with any of the previous catheters. The length s of septum 18 is larger than a maximum distance between two points on inner surface 14 of wall 15.

In the embodiment shown in FIGS. 4A and 4B, septum 18 is formed from a material that is both flexible and elastic. Fluid flowing in lumen 24 exerts a force on septum 18 which displaces septum 18 from its equilibrium position. As shown in FIG. 4B, under a sufficiently large applied force, septum 18 can contact a portion of inner surface 14 of wall 15 in lumen 26. The length s′ of stretched septum 18 in FIG. 4B is greater than s. Displacement of septum 18 as shown in FIG. 4B leads to a large increase in the cross-sectional area of lumen 24, and a corresponding decrease in the cross-sectional area of lumen 26. Lumen 24 therefore supports a larger fluid flow rate, while lumen 26 supports a reduced fluid flow rate, relative to FIG. 4A. In certain embodiments, lumen 26 can be sealed by septum 18. Lumen 26 re-opens when the fluid pressure is reduced in lumen 24, and elastic forces in septum 18 pull the septum material away from inner wall 14 in lumen 26.

In some embodiments, one or more anti-adhesion coatings can be applied to inner surfaces of catheters to reduce or prevent adhesion of septum 18 to inner surface 14 of wall 15. FIG. 5 shows a catheter 400 that includes an anti-adhesion coating 40 applied to inner surface 14 of wall 15 in lumens 24 and 26. Anti-adhesion coating 40 has a thickness t_{c} measured in a radial direction perpendicular to axis 12. In general, anti-adhesion coating 40 can be formed only on selected inner surfaces of catheter 400, or anti-adhesion coating 40 can be formed on all inner surfaces of catheter 400 as shown in FIG. 5.

Anti-adhesion coating 40 can be formed from a variety of materials. For example, anti-adhesion coating 40 can include Teflon®-based materials, other fluoropolymer-based materials, and other anti-adhesive materials. Examples of anti-adhesive materials include silicone, parylene, MediGlide™ and BioSlide™. In certain embodiments, coating 40 can include more than one material and/or multiple layers of different materials.

The thickness t_{c} of anti-adhesion coating 40 can generally vary as desired. For example, in some embodiments, t_{c} can be 0.003 inch or more (e.g., 0.004 inch or more, 0.005 inch or more, 0.007 inch or more, 0.01 inch or more). In certain embodiments, t_{c} can be 0.05 inch or smaller (e.g., 0.02 inch or smaller, 0.01 inch or smaller, 0.005 inch or smaller, 0.001 inch or smaller).

In some embodiments, wall 15 can include protrusions configured to reduce or prevent adhesion of septum 18 to inner surface 14 of wall 15. FIG. 6A shows a catheter 500 that includes a plurality of protrusions 50 that extend radially inward toward the interior of catheter 500. Protrusions 50 have a maximum length h measured in a radial direction perpendicular to axis 12. Protrusions 50 have a maximum width w measured along the circumferential direction of catheter 500, and are spaced a distance p apart measured along a circumferential direction of catheter 500. Protrusions 50 are formed from the same material as wall 15, and can also be provided with an anti-adhesion coating 40 as discussed previously.

The maximum length h of protrusions 50 can generally be selected as desired to control a maximum displacement of septum 18, and therefore to control the cross-sectional shapes of lumens 24 and 26 when septum 18 is displaced. For example, in some embodiments, h can be 0.003 inch or more (e.g., 0.004 inch or more, 0.005 inch or more, 0.007 inch or more, 0.01 inch or more). In certain embodiments, h can be 0.05 inch or smaller (e.g., 0.02 inch or smaller, 0.01 inch or smaller, 0.005 inch or smaller, 0.001 inch or smaller). In certain embodiments, the maximum length h of protrusions 50 is less than the thickness t_{w} of wall 15. In some embodiments, a ratio of h/t_{w} is at least 0.05:1 (e.g., at least 0.1:1, at least 0.2:1, at least 0.3:1, at least 0.4:1, at least 0.5:1). In certain embodiments, h/t_{w} is at most 0.95:1 (e.g., at most 0.9:1, at most 0.8:1, at most 0.7:1, at most 0.6:1, at most 0.5:1).

The maximum width w of protrusions 50 can generally be selected as desired to control the surface area of contact between the protrusions and septum 18. In some embodiments, w can be 0.001 inch or more (e.g., 0.002 inch or more, 0.003 inch or more, 0.005 inch or more, 0.007 inch or more). In certain embodiments, w can be 0.05 inch or less (e.g., 0.03 inch or less, 0.01 inch or less, 0.009 inch or less, 0.008 inch or less). In certain embodiments, the ratio of w to h can be at least 0.1:1 (e.g., at least 0.5:1, at least 1:1), and/or at most 10:1 (e.g., at most 5:1, at most 1:1).

The spacing p between protrusions 50 is selected to control the total surface area of contact between the protrusions and septum 18. A larger spacing p corresponds to a smaller number of protrusions 50 in catheter 500, and a greater likelihood (due to the smaller protrusion density) that septum 18 will contact a portion of inner surface 14 of wall 15. A smaller spacing p corresponds to a larger number of protrusions 50 in catheter 500, and a lesser likelihood (due to the larger protrusion density) that septum 18 will contact a portion of inner surface 14 of wall 15. In general, spacing p can be selected as desired. In some embodiments, p can be 0.005 inch or more (e.g., 0.006 inch or more, 0.007 inch or more, 0.008 inch or more, 0.01 inch or more, 0.03 inch or more, 0.05 inch or more). In certain embodiments, p can be 0.1 inch or less (e.g., 0.09 inch or less, 0.08 inch or less, 0.07 inch or less, 0.05 inch or less).

Generally, the number of protrusions 50 can also be selected as desired. For example, in some embodiments, there can be one or more (e.g., two or more, three or more, four or more, five or more) protrusions 50, and or 20 or less (e.g., 15 or less, 10 or less) or protrusions 50.

When fluid flows in a lumen of catheter 500, septum 18 is displaced from its equilibrium position, as discussed previously. If septum 18 is formed from a material that is flexible and elastic, fluid pressure can displace septum 18 so that, in the absence of protrusions 50, septum 18 would contact a portion of inner surface 14 of wall 15. However, as shown in FIG. 6B, protrusions 50 can reduce and/or prevent septum 18 from contacting inner wall 14. The area of contact between the surfaces of protrusions 50 and septum 18 can be smaller than the area of contact between septum 18 and inner surface 14 would be, due to the shapes of protrusions 50. As a result, adhesion forces between septum 18 and protrusions 50 are not as large as adhesion forces between septum 18 and inner surface 14 would otherwise be. Septum 18 is therefore more easily pulled away from the surfaces of protrusions 50 by elastic forces in septum 18 when the fluid pressure is reduced in lumen 24.

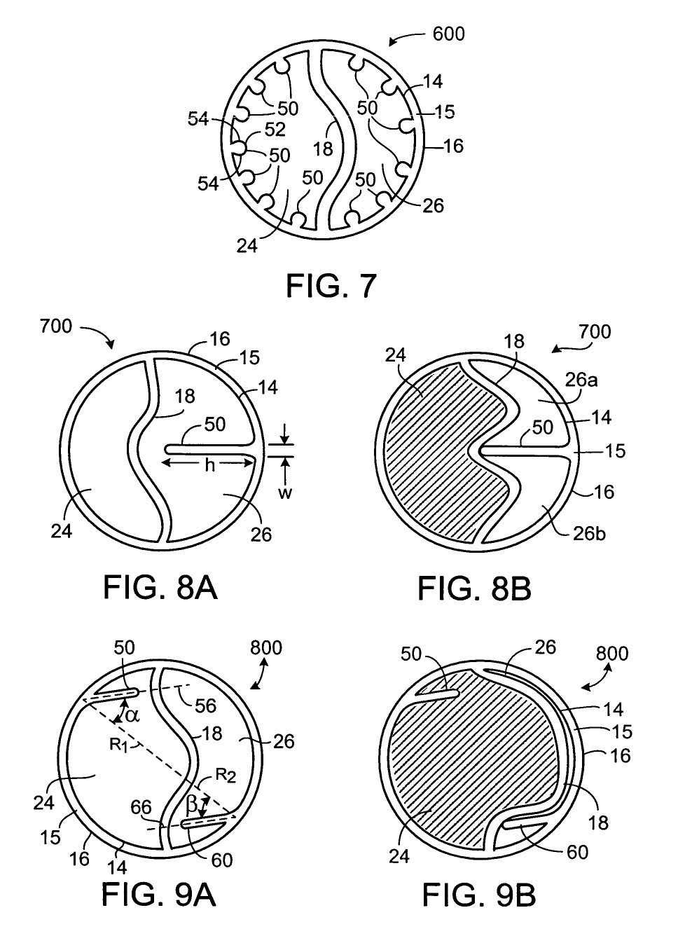

The cross-sectional shapes of protrusions 50 can generally be selected as desired to prevent adhesion of septum 18 to inner wall 14. A variety of different shapes are possible, including arc segments, half-round shapes. rectangular shapes, triangular shapes, trapezoidal shapes, and other shapes. In certain embodiments, cross-sectional shapes with an undercut (e.g., a 270° arc segment) are advantageous because septum 18 does not generally adhere to undercut portions of a protrusion. As a result, undercut cross-sectional shapes may reduce even further a surface area of contact between septum 18 and protrusions 50. An example of a catheter 600 with protrusions 50 having undercut regions is shown in FIG. 7. Protrusions 50 have shapes which are similar to arc segments of 270°. When displaced by fluid pressure from its equilibrium position, septum 18 contacts protrusions 50 in regions 52. However, septum 18 generally does not contact protrusions 50 in undercut regions 54. As a result, adhesive forces between septum 18 and protrusions 50 occur only in regions 52 of the protrusion surfaces.

In some embodiments, catheters can include fewer protrusions than catheter 500 in FIGS. 6A and 6B. FIGS. 8A and 8B show side views of a catheter 700 without and with fluid flowing in lumen 24, respectively. Catheter 700 includes a single protrusion 50 that extends radially inward toward the center of catheter 700. Protrusion 50 can be formed from any of the materials discussed previously in connection with protrusions.

The length h of protrusion 50 is generally less than half the length s of septum 18, and can be selected as desired. The width w of protrusion 50 is generally selected according to a desired stiffness for protrusion 50. That is, w is chosen to be larger to provide a protrusion 50 that is stiffer and less compliant with respect to deformation under an applied load, and w is chosen to be smaller to provide a protrusion 50 that is less stiff and more compliant with respect to deformation.

In the embodiment shown in FIGS. 8A and 8B, protrusion 50 is a relatively stiff protrusion that resists deformation. When fluid flows in lumen 24, septum 18 is displaced so that it contacts a portion of protrusion 50 as shown in FIG. 8B. Protrusion 50 reduces or prevents contact between septum 18 and inner wall 14 of lumen 26. As a result of the displacement of septum 18, lumen 26 is divided into two smaller lumens 26a and 26b. The properties of both septum 18 and protrusion 50 determine the cross-sectional shapes of lumens 24 and 26 when septum 18 is displaced from its equilibrium position (shown in FIG. 8A). For example, the length h of protrusion 50 in FIG. 8A determines the maximum displacement of septum 18 near the center of catheter 700. When the fluid pressure in lumen 24 is reduced, septum 18 returns to its equilibrium position.

In certain embodiments, protrusions can be oriented at an angle to the radial direction of the catheter (e.g., at an angle to a direction perpendicular to axis 12). FIGS. 9A and 9B show side views of a catheter 800 without and with fluid flowing in lumen 24, respectively. Catheter 800 includes a first protrusion 50 and a second protrusion 60. First protrusion 50 has a longitudinal axis 56 that is oriented at an angle α to a radial direction line indicated by R₁. In general, a range from 1° or less to 179° or more. In some embodiments, α can be 1° or more (e.g., 5° or more, 10° or more, 20° or more, 30° or more). In certain embodiments, α can be 89° or less (e.g., 85° or less, 80° or less, 70° or less, 60° or less, 50° or less). Second protrusion 60 has a longitudinal axis 66 that is oriented at an angle β with respect to a radial direction line indicated by R₂. In general, β can have any of the values discussed in connection with α. In certain embodiments, β and α have similar values. In other embodiments, the values of β and α are different.

In general, catheter 800 can include more than two protrusions (e.g., three or more protrusions, four or more protrusions, five or more protrusions, ten or more protrusions). The protrusions can be symmetrically positioned along inner surface 14 of wall 15, or they can be asymmetrically positioned. Protrusions can further be oriented along radial directions of catheter 800 or along non-radial directions, as desired. The dimensions of protrusions (e.g., maximum length h and maximum width w) and the cross-sectional shapes of protrusions in catheter 800 can all be the same, or some of the protrusions can have different dimensions and/or cross-sectional shapes.

When fluid flows in lumen 24, as shown in FIG. 9B, septum 18 (which is formed from a flexible and elastic material) is displaced from its equilibrium position. Protrusion 60 reduces a surface area of contact between septum 18 and inner surface 14 of wall 15 in lumen 26. The angled orientation of protrusion 60 with respect to R₂ provides a larger cross-sectional area of lumen 24 when septum 18 is displaced, compared to the radially-oriented protrusion 50 in FIG. 8B, for example. Protrusion 50 in FIG. 9A operates in similar fashion to protrusion 60 when fluid is directed to flow in lumen 26. In general, protrusions oriented along directions other than radial directions of a catheter can be used to increase the cross-sectional area of fluid-carrying lumens when septum 18 is displaced from its equilibrium position.

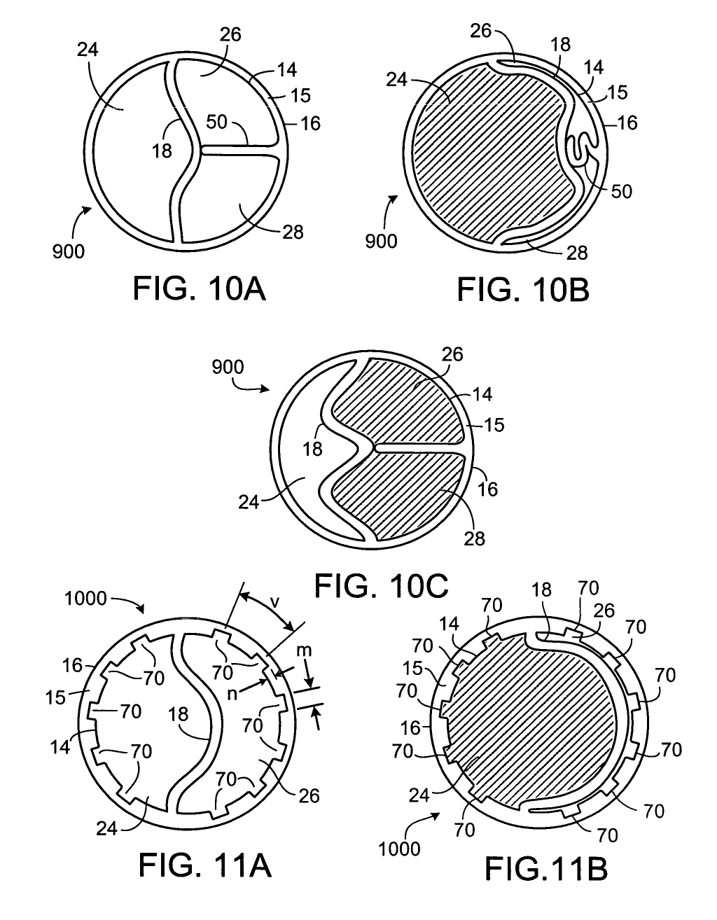

In some embodiments, protrusions can be attached to the septum of a catheter. FIGS. 10A-C show side views of a catheter 900 without fluid therein, with fluid flowing in lumen 24, and with fluid flowing in lumens 26 and 28, respectively. Catheter 900 includes a septum 18 and a protrusion 50 that are each formed from flexible and elastic materials. Protrusion 50 is attached to septum 18, so that catheter 900 includes three lumens 24, 26, and 28. When fluid flows through lumen 24, as shown in FIG. 10B, septum 18 is displaced from its equilibrium position. Under the force applied by fluid pressure via septum 18, protrusion 50 folds against inner surface 14 of wall 15. Protrusion 50 reduces or prevents contact between septum 18 and inner surface 14. In addition, due to the folded geometry of protrusion 50, the cross-sectional area of lumen 24 is larger than it would otherwise be if protrusion 50 did not fold (see for example FIG. 8B). When the fluid pressure in lumen 24 is reduced, septum 18 returns toward its equilibrium position. Both elastic forces in septum 18 (which pull the septum material back toward the equilibrium position) and compression forces in folded protrusion 50 (which push the septum material away from wall 15 and toward the equilibrium position) assist in returning septum 18 to its original position.

Fluid can also flow in either or both of lumens 26 and 28. In certain embodiments, lumens 26 and 28 are coupled to the same fluid source. In other embodiments, lumens 26 and 28 are coupled to different fluid sources. As shown in FIG. 10C, when fluid flows in lumens 26 and/or 28, septum 18 is displaced from its equilibrium position in the direction of inner surface 14 of wall 15 in lumen 24. Contact between inner surface 14 and septum 18 in lumen 24 can be reduced or prevented by protrusion 50. As septum 18 is displaced further from its equilibrium position, protrusion 50 can become elongated. Elastic forces in both septum 18 and protrusion 50 pull septum 18 back toward its equilibrium position and away from inner surface 14 of wall 15 in lumen 24. By balancing the elastic forces in septum 18 and protrusion 50 against the pressure applied by fluid in lumens 26 and/or 28, contact between septum 18 and inner surface 14 in lumen 24 can be reduced or avoided. The cross-sectional area of each of lumens 26 and 28 in FIG. 10C is larger than the cross-sectional area of these lumens if protrusion 50 were formed from an inelastic material (e.g., a material that did not stretch significantly in response to applied fluid pressure).

In general, multiple protrusions attached to septum 18 can be provided in catheters. In some embodiments, a mixture of elastic and inelastic protrusions can be provided. In other embodiments, all of the protrusions can be either elastic or inelastic. By attaching the protrusions to septum 18, multiple lumens can be formed in a catheter (e.g., two or more lumens, three or more lumens, four or more lumens, five or more lumens, six or more lumens, ten or more lumens).

In certain embodiments, catheters can include one or more recesses extending radially outward from an inner surface of the catheter wall. FIGS. 11A and 11B show side views of a catheter 1000 without and with fluid flowing in lumen 24, respectively. Catheter 1000 includes a plurality of recesses 70 extending radially outward from inner surface 14 of wall 15. Recesses 70 have a maximum depth n measured in a radial direction perpendicular to axis 12. Recesses 70 have a maximum width m and are spaced at a distance v apart, both measured in a circumferential direction of catheter 1000.

Recesses 70 reduce the surface area of contact between septum 18 and inner surface 14 of wall 15. As shown in FIG. 11B, when a fluid flows in lumen 24, septum 18 (which is formed from a flexible and elastic material) is displaced from its equilibrium position by fluid pressure. If the force applied to septum 18 by the fluid in lumen 24 is sufficiently large, septum 18 can stretch and contact a portion of inner surface 14 of wall 15 in lumen 26. Adhesion forces can be present between septum 18 and inner surface 14 which hinder the return of septum 18 to its equilibrium position when the fluid pressure in lumen 24 is reduced. By reducing the surface area of contact between septum 18 and inner surface 14 via recesses 70, the magnitude of the adhesion forces is reduced, and elastic forces within septum 18 can pull the septum material away from inner surface 14.

The dimensions of recesses 70 can generally be selected as desired to reduce the surface area of contact between septum 18 and inner surface 14 of wall 15. In some embodiments, the spacing v can be 0.005 inch or more (e.g., 0.006 inch or more, 0.007 inch or more, 0.008 inch or more, 0.01 inch or more, 0.03 inch or more, 0.05 inch or more). In certain embodiments, the spacing v can be 0.1 inch or less (e.g., 0.09 inch or less, 0.08 inch or less, 0.07 inch or less, 0.05 inch or less).

In some embodiments, the maximum width m of recesses 70 can be 0.001 inch or more (e.g., 0.002 inch or more, 0.003 inch or more, 0.005 inch or more, 0.007 inch or more). In certain embodiments, the maximum width m of recesses 70 can be 0.05 inch or less (e.g., 0.03 inch or less, 0.01 inch or less, 0.009 inch or less, 0.008 inch or less).

The maximum depth n of recesses 70 can generally be selected as desired. For example, in some embodiments, n can be 0.003 inch or more (e.g., 0.004 inch or more, 0.005 inch or more, 0.007 inch or more, 0.01 inch or more). In certain embodiments, n can be 0.05 inch or smaller (e.g., 0.02 inch or smaller, 0.01 inch or smaller, 0.005 inch or smaller, 0.001 inch or smaller). Typically, the maximum depth n of recesses 70 is less than the thickness t_{w} of wall 15. In some embodiments, a ratio of n/t_{w} is at least 0.05:1 (e.g., at least 0.1:1, at least 0.2:1, at least 0.3:1, at least 0.4:1, at least 0.5:1). In certain embodiments, n/t_{w} is at most 0.95:1 (e.g., at most 0.9:1, at most 0.8:1, at most 0.7:1, at most 0.6:1, at most 0.5:1).

The cross-sectional shapes of recesses 70 can generally be selected as desired to reduce the surface area of contact between septum 18 and inner surface 14 of wall 15. In catheter 1000, as shown in FIGS. 11A and 11B, recesses 70 have approximately rectangular cross-sectional shapes. In general, however, recesses 70 can have a variety of cross-sectional shapes. For example, recesses 70 can be arc-shaped (e.g., corresponding to circular arcs), triangular-shaped, trapezoid-shaped, or other symmetric or asymmetric shapes. In certain embodiments, recesses 70 all have the same cross-sectional shape. In other embodiments, at least some of recesses 70 have different cross-sectional shapes.

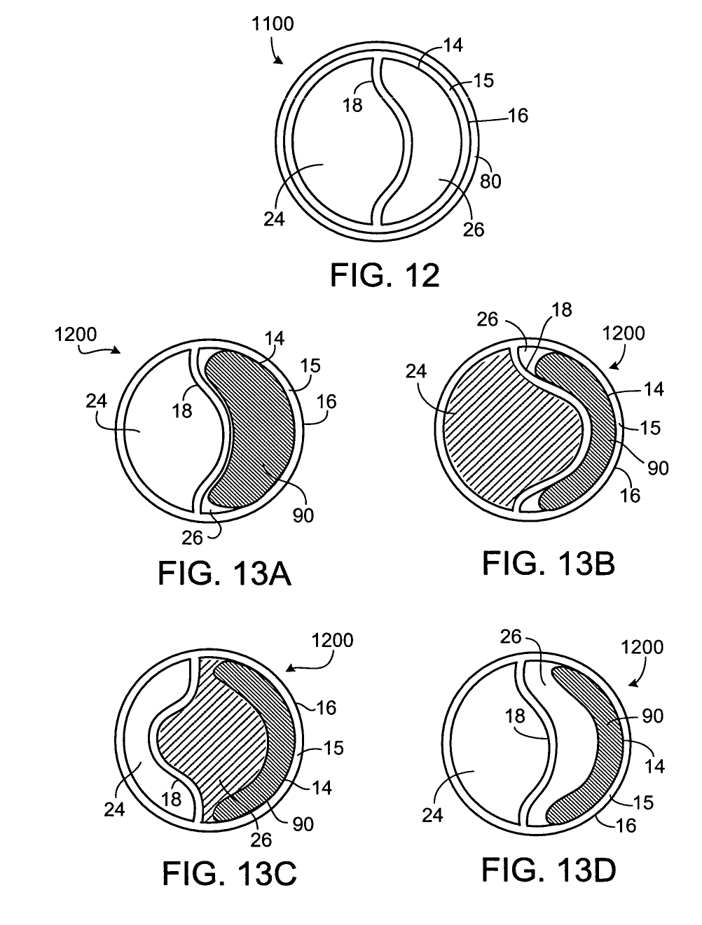

In some embodiments, catheters can include an outer layer of material to restrict expansion of the catheter. FIG. 12 shows an embodiment of a catheter 1100 that includes a coating 80 disposed on outer surface 16 of wall 15. Wall 15 is formed from a material that is at least partially elastic, e.g., wall 15 stretches at least partially in response to fluid pressure in either or both of lumens 24 and 26. In many applications, however, catheters are inserted into small diameter body lumens such as veins and arteries which do not allow for significant radial expansion of the catheter. If the catheter expands radially in response to fluid flow therein, damage to the body lumen can result. To prevent radial expansion of catheter 1100, coating 80 surrounds outer surface 16. Coating 80 is formed from an inelastic material that does not stretch or deform appreciably in response to fluid pressure in lumens 24 and/or 26.

Coating 80 can be formed from a variety of known materials. Optionally, coating 80 can be formed via a coextrusion process.

Catheters positioned at body sites (e.g., within body lumens) can become occluded during use due to precipitates and other debris carried by fluids flowing therein. In some embodiments, an occlusion in one lumen of a multiple-lumen catheter can be reduced in size or cleared by manipulating the other lumens in the catheter. FIGS. 13A-D show a series of steps that can be performed to reduce the size of an occlusion in one lumen of a two-lumen catheter 1200. Catheter 1200 includes a first lumen 24 and a second lumen 26 with an occlusion 90 present therein. Occlusion 90, as shown in FIG. 13A, significantly reduces the open cross-sectional area of lumen 26, thereby reducing the fluid carrying capacity of lumen 26. To reduce the size of occlusion 90, a fluid is flowed at a relatively high pressure through lumen 24, as shown in FIG. 13B. Septum 18 is displaced from its equilibrium position in a direction toward lumen 26. Septum 18 compacts occlusion 90 against inner surface 14 of wall 15 in lumen 26. Fluid flow in lumen 24 is then halted and fluid flow in lumen 26 is initiated, as shown in FIG. 13C. The pressure exerted by the fluid in lumen 26 and the elastic forces in septum 18 displace septum 18 in the direction of lumen 24, opening lumen 26 in the process. Once lumen 26 is re-opened, fluid flow therein is halted and septum 18 returns to its equilibrium position, as shown in FIG. 13D. Occlusion 90 may remain adhered to inner surface 14 of wall 15 in lumen 26 because the materials which form occlusions can be sticky biological materials. However, the cross-sectional area of occlusion 90 is reduced relative to FIG. 13A, and the open cross-sectional area of lumen 26 is increased relative to FIG. 13A, which re-enables the use of lumen 26 for various fluid transport operations.

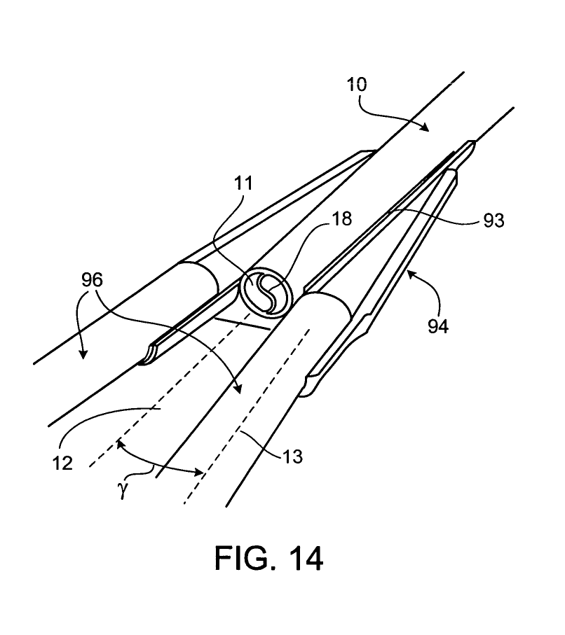

The lumens in a multiple-lumen catheter can generally be attached to separate outlet tubes in a hub. Various types of hubs can be used to securely connect a catheter and outlet tubes. A portion of one such hub is shown in FIG. 14. The hub includes a support base 94 and a top (not shown) which snaps into secure engagement with support base 94. Catheter 10, which includes two lumens separated by septum 18, is positioned at one end of support base 94. Access slits 93 are provided in the wall of catheter 10 to provide fluid access to the two catheter lumens. Two outlet tubes 96 are positioned such that one tube is in fluid connection with one of the catheter lumens and the other tube is in fluid connection with the other catheter lumen. To complete the fluid connections and close the hub, the top (not shown) is snapped into engagement with support base 94 and end 11 of catheter 10 is sealed with a casting material (e.g., a polymer material such as Carbothane™). In some embodiments, the hub can be overmolded with casting material to further secure the connections between catheter 10 and outlet tubes 96.

Outlet tubes 96 each have a longitudinal axis 13. The outlet tubes are positioned relative to catheter 10 in the hub so that axis 13 is oriented at an angle γ with respect to axis 12 of catheter 10. In general, γ can be chosen to control an amount of lateral force applied to septum 18 (e.g., force applied in a direction perpendicular to the surface of septum 18). For example, for large angles γ, a relatively large amount of lateral force is applied to septum 18 by fluids flowing into catheter 10 from outlet tubes 96. For small angles γ, a relatively small amount of lateral force is applied to septum 18 by fluids flowing into catheter 10 from outlet tubes 96. Because lateral force results in displacement of septum 18 from its equilibrium position, the ease with which lumen cross-sectional areas in catheter 10 can be enlarged can be controlled by selecting appropriate angles γ. In the embodiment shown in FIG. 14, axis 13 of each outlet tube 96 forms a similar angle γ with axis 12. However, in other embodiments, the axes 13 of outlet tubes 96 can form different angles with axis 12, according to desired performance criteria for catheter 10.

In certain embodiments, for example, γ can be 3° or more (e.g., 4° or more, 5° or more, 10° or more, 20° or more). In some embodiments, γ can be 70° or less (e.g., 65° or less, 60° or less, 50° or less).

Different features of medical devices such as catheters have been disclosed above. Embodiments can, in general, include any of the disclosed features, as appropriate, to produce medical devices that achieve particular functional and/or performance criteria.

Many different types of catheters can include multiple internal lumens. For example, the catheters disclosed herein can be ocular shunts, endoscopic catheters, peripherally inserted catheters, dialysis catheters, PTA catheters, angiography catheters, drainage catheters, PTCA catheters, overall venous access devices (e.g., tunneled central catheters, midline catheters, subcutaneous port catheters) and other types of catheters.

Multiple-lumen features can also be provided in various types of stents. For example, coronary stents, aortic stents, peripheral vascular stents, gastrointestinal stents, urinary stents, and neurology stents can include the multiple-lumen features disclosed herein. As an example, urinary stents can become occluded with deposits carried by urinary fluids, and the cross-sectional area of the occlusions can be reduced in multiple-lumen urinary stents using the techniques shown in FIGS. 13A-D.

Other embodiments are in the claims.

What is claimed is:

1. An article, comprising:
an elongated tubular element having a wall, the wall having an inner surface; and
a septum attached to the inner surface of the wall in at least two places so that the septum and wall form at least two lumens in the catheter,
wherein a length of the septum is greater than or equal to a maximum distance between two points on the inner surface of the wall, and the article is a catheter.

2. The article of claim 1, wherein the septum is attached to the inner surface of the wall in two places.

3. The article of claim 1, wherein a ratio of the length of the septum to the maximum distance between two points on the inner surface of the wall is at least 1:1.

4. The article of claim 1, wherein a ratio of the length of the septum to the maximum distance between two points on the inner surface of the wall is at most 10:1.

5. The article of claim 1, wherein the septum comprises a polymer.

6. The article of claim 1, further comprising an anti-adhesive material supported by the inner surface of the wall.

7. The article of claim 1, further comprising a material supported by the outer surface of the wall.

8. The article of claim 1, wherein the wall includes a protrusion that extends radially inward.

9. The article of claim 1, wherein the wall has a recess that extends radially outward.

10. An article, comprising:
an elongated tubular element having a wall, the wall having an inner surface;
a septum attached to the inner surface of the wall in at least two places so that the septum and wall form at least two lumens in the catheter; and
an anti-adhesive material supported by the inner surface of the wall,
wherein the article is a catheter.

11. The article of claim 10, wherein the septum is attached to the inner surface of the wall in two places.

12. The article of claim 10, wherein the anti-adhesive material is on the inner surface of the wall.

13. The article of claim 10, further comprising a material supported by the outer surface of the wall.

14. The article of claim 10, wherein the wall includes a protrusion that extends radially inward.

15. The article of claim 10, wherein the wall has a recess that extends radially outward.

16. An article, comprising:
an elongated tubular element having a wall, the wall having an inner surface and an outer surface;
a septum attached to the inner surface of the wall in at least two places so that the wall and the septum form at least two lumens; and
a material supported by the outer surface of the wall,
wherein the article is a catheter.

17. The article of claim 16, wherein the septum is attached to the inner surface of the wall in two places.

18. The article of claim 16, wherein the material is on the outer surface of the wall.

19. The article of claim 16, wherein the wall includes a protrusion that extends radially inward.

20. The article of claim 16, wherein the wall has a recess that extends radially outward.

21. An article, comprising:
an elongated tubular element having a wall, the wall having an inner surface, the wall including a protrusion that extends radially inward; and
a septum attached to the inner surface of the wall in at least two places so that the wall and the septum form at least two lumens,
wherein the article is a catheter.

22. The article of claim 21, wherein the septum is attached to the inner surface of the wall in two places.

23. The article of claim 21, wherein a length of the protrusion is less than half a length of the septum.

24. The article of claim 21, wherein the wall has a recess that extends radially outward.

25. An article, comprising:
an elongated tubular element having a wall, the wall having an inner surface, the wall having a recess that extends radially outward; and
a septum attached to the inner surface of the wall in at least two places,
wherein the article is a catheter.

26. The article of claim 25, wherein the septum is attached to the inner surface of the wall in two places

27. The article of claim 25, wherein a ratio of a depth of the recess to a maximum thickness of the wall is at most 0.95:1.

28. The article of claim 25, wherein a ratio of a depth of the recess to a maximum thickness of the wall is at least 0.05:1.
